(19) United States
(12) Patent Application Publication
(10) Pub. No.: US 2025/0072976 A1
(43) Pub. Date: Mar. 6, 2025

(54) AUGMENTED REALITY SYSTEM AND AUGMENTED REALITY METHOD

(71) Applicant: NATIONAL TAIWAN UNIVERSITY, TAIPEI (TW)

(72) Inventors: Min-Hung LO, TAIPEI (TW); Hao-Li LIU, TAIPEI (TW)

(21) Appl. No.: 18/616,624

(22) Filed: Mar. 26, 2024

(30) Foreign Application Priority Data
Sep. 5, 2023 (TW) 112133744

(51) Int. Cl. A61B 34/20; A61B 90/00; G06T 7/50; G06T 7/73

(52) U.S. Cl. CPC A61B 34/20; A61B 90/37; G06T 7/50; G06T 7/74; A61B 2034/2055; A61B 2034/2063; A61B 2090/365; G06T 2207/30004; G06T 2207/30204

(57) ABSTRACT

The augmented reality system includes a needle, a camera, an ultrasound generator, a memory, and a processor. The needle includes a location code, a first marking area, and a second marking area. The processor is used to perform following steps: capturing the location code, the first marking area, and the second marking area of the needle to perform positioning or a marking pose estimation by the camera; obtaining a feature points number according to the first marking area and the second marking area; obtaining a range prediction interval of the ultrasound field of view according to the feature points number of the needle; increasing or decreasing the range prediction interval according to the feature points number and a feature points number threshold; and when it is determined that the feature points number is greater than the feature points number threshold, the range prediction interval is reduced.

### CROSS-REFERENCE TO RELATED APPLICATION

This application claims priority to Taiwan Application Serial Number 112133744, filed Sep. 5, 2023, which is herein incorporated by reference in its entirety.

### BACKGROUND

### Field of Invention

The present invention relates to a reality system and reality method. More particularly, the present invention relates to an augmented reality system and augmented reality method.

### Description of Related Art

Currently, needle is mostly used in medical treatment for radiofrequency ablation, and different forms of location codes (or markers) are often used to locate the needle position.

However, when capturing the location code on the needle, the limiting camera field of view makes it easy for the location code to move out of the camera's detectable range during the operation, causing positioning to be terminated.

Therefore, how to enable the needle to continue positioning when the location code is not within the limiting camera field of view is actually a problem that the industry urgently needs research and development breakthrough.

### SUMMARY

The present disclosure provides an augmented reality system. The augmented reality system includes a needle, a camera, an ultrasound generator, a memory, and a processor. The needle includes a location code, a first marking area, and a second marking area. The first marking area has a first grayscale color. The second marking area has a second grayscale color, the first grayscale color and the second grayscale color are different from each other, and the second grayscale color is arranged after the first marking area. The ultrasound generator is used to form an ultrasound field of view based on a measuring object. The memory is used to store a plurality of commands. The processor is used to perform following steps according to the plurality of commands of the memory: capturing the location code, the first marking area, and the second marking area of the needle to perform positioning or a marking pose estimation by the camera; confirming the first marking area and the second marking area of the needle are located on a straight line for a feature pose estimation according to a linear regression algorithm; comparing the first marking area, the second marking area, and a matching template to confirm a direction of the needle; obtaining a feature points number according to the first marking area and the second marking area, wherein the feature points number is a positive integer greater than or equal to 2; obtaining a range prediction interval of the ultrasound field of view according to the feature points number of the needle; increasing or decreasing the range prediction interval according to the feature points number and a feature points number threshold; and when it is determined that the feature points number is greater than the feature points number threshold, the range prediction interval is reduced.

The present disclosure provides an augmented reality method. The augmented reality method includes the following steps: forming an ultrasound field of view based on a measuring object by an ultrasound generator; capturing a location code, a first marking area, and a second marking area of a needle to perform positioning or a marking pose estimation by a camera, wherein the first marking area has a first grayscale color, the second marking area has a second grayscale color, the first grayscale color and the second grayscale color are different from each other, and the second grayscale color is arranged after the first marking area; confirming the first marking area and the second marking area of the needle are located on a straight line for a feature pose estimation by a processor according to a linear regression algorithm; comparing the first marking area, the second marking area, and a matching template to confirm a direction of the needle by the processor; confirming the first marking area and the second marking area of the needle are located on the straight line for a needle bending detection by the processor according to at least one of the linear regression algorithm and a bending relational expression; obtaining a feature points number according to the first marking area and the second marking area by the processor, wherein the feature points number is a positive integer greater than or equal to 2; obtaining a range prediction interval of the ultrasound field of view by the processor according to the feature points number of the needle; increasing or decreasing the range prediction interval by the processor according to the feature points number and a feature points number threshold; and when it is determined that the feature points number is greater than the feature points number threshold by the processor, the range prediction interval is reduced.

Therefore, according to the technical content of the present disclosure, the augmented reality system and the augmented reality method shown in the embodiment of the present disclosure can achieve the effect of expanding the field off view by detecting (or capturing) a plurality of marking areas and a plurality of feature points on the needle.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a block diagram of an augmented reality system according to one embodiment of the present disclosure.

FIG. 2 is a usage scenarios diagram of an augmented reality system according to one embodiment of the present disclosure.

FIG. 3A is a usage scenarios diagram of an augmented reality system according to one embodiment of the present disclosure.

FIG. 3B is a usage scenarios diagram of an augmented reality system according to one embodiment of the present disclosure.

FIG. 3C is a usage scenarios diagram of an augmented reality system according to one embodiment of the present disclosure.

FIG. 4 is a usage scenarios diagram of an augmented reality system according to one embodiment of the present disclosure.

FIGS. 5A to 5C are usage scenarios diagrams of an augmented reality system according to one embodiment of the present disclosure.

FIGS. 6A to 6C are usage scenarios diagrams of an augmented reality system according to one embodiment of the present disclosure.

FIGS. 7A to 7C are usage scenarios diagrams of an augmented reality system according to one embodiment of the present disclosure.

FIGS. 8A to 8C are usage scenarios diagrams of an augmented reality system according to one embodiment of the present disclosure.

FIGS. 9A to 9B are step flowing diagrams of an augmented reality method according to one embodiment of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

The embodiments below are described in detail with the accompanying drawings, but the examples provided are not intended to limit the scope of the disclosure covered by the description. The structure and operation are not intended to limit the execution order. Any structure regrouped by elements, which has an equal effect, is covered by the scope of the present disclosure.

Various embodiments of the present technology are discussed in detail below with figures. It should be understood that the details should not limit the present disclosure. In other words, in some embodiments of the present disclosure, the details are not necessary. In addition, for simplification of figures, some known and commonly used structures and elements are illustrated simply in figures.

In the present disclosure, “connected” or “coupled” may refer to “electrically connected” or “electrically coupled.” “Connected” or “coupled” may also refer to operations or actions between two or more elements.

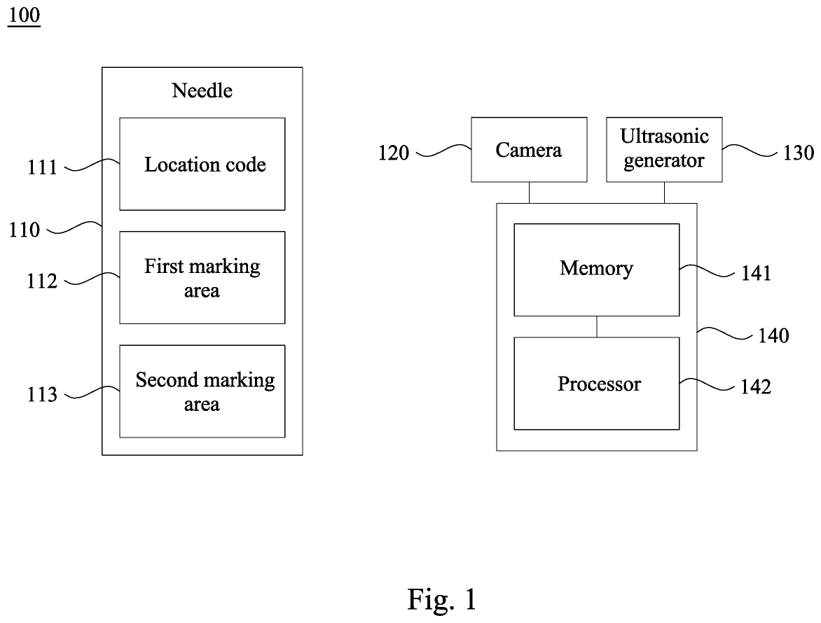

FIG. 1 is a block diagram of an augmented reality system according to one embodiment of the present disclosure. As shown in FIG. 1, in some embodiments, the augmented reality system 100 includes a needle 110, a camera 120, an ultrasound generator 130, and a host 140. The host 140 includes a memory 141, and a processor 142. The needle includes a location code 111, a first marking area 112, and a second marking area 113.

Regarding a coupling relationship, the camera 120 is coupled to the host 140, the ultrasound generator 130 is coupled to the host 140. Within the host 140, the memory 141 is coupled to the processor 142.

For example, the needle 110 can be a needle which is used for a radiofrequency ablation (RFA) or a biological sampling. The location code 111 can be a two-dimensional maker (such as a maker diamond aruco), the location code 111 can be fixed on a handle of the needle 110, and the location code 111 quickly locates the two-dimensional coordinates on the two-dimensional mark to the known three-dimensional object specifications through the image processing algorithm (such as a software OpenCV), but the present disclosure is not limited to this embodiment.

In some embodiments, the first marking area 112 has a first grayscale color. The second marking area 113 has a second grayscale color. The first grayscale color and the second grayscale color are different from each other, and the second grayscale color 113 is arranged after the first marking area 112.

For example, the first marking area 112 can be a rectangular area, the first grayscale color can be a grayscale of white, gray, black, or other colors. The second marking area 113 can be the rectangular area, the second grayscale color can be the grayscale of white, gray, black, or other colors. When the first grayscale color is black, the second grayscale color can be white, but the present disclosure is not limited to this embodiment.

In some embodiments, the ultrasound generator 130 is used to form an ultrasound field of view based on a measuring object. The memory 141 is used to store a plurality of commands.

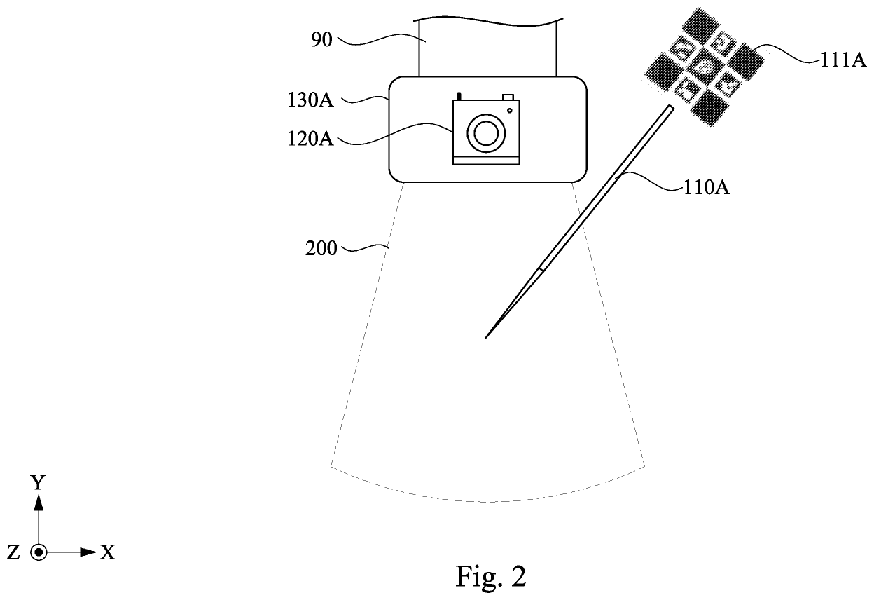

For example, the ultrasound generator 130 can be an ultrasound probe, the measuring object can be a human body or an organism, and the ultrasound field of view can be an image produced by an ultrasound passing through the human body or the organism (as shown in FIG. 2, an ultrasound field of view 200). The memory 141 can be a storage hardware (such as an Electrically-Erasable Programmable Read-Only Memory (EEPROM) or a flash memory). The plurality of commands can be computer readable language or application language, but the present disclosure is not limited to this embodiment.

FIG. 2 is a usage scenarios diagram of an augmented reality system according to one embodiment of the present disclosure. As shown in FIG. 2, in some embodiments, users can place the camera 120A and the ultrasound generator 130A on the measuring object 90, a position of the camera 120A and the ultrasound generator 130A can be regarded as an initial position (such as coordinate (X,Y) can be (0,0)). The needle 110A has the location code 111A, the camera 120A is used to capture the location code 111A to perform positioning of the needle 110A, and the ultrasound generator 130A is used to generate the ultrasound and the ultrasound produces the ultrasound field of view 200 through the measuring object 90.

In some embodiments, the needle 110A in FIG. 2 corresponds to the needle 110 in FIG. 1, the location code 111A in FIG. 2 corresponds to the location code 111 in FIG. 1, the camera 120A in FIG. 2 corresponds to the camera 120 in FIG. 1, the ultrasound generator 130A in FIG. 2 corresponds to the ultrasound generator 130 in FIG. 1, but the present disclosure is not limited to this embodiment.

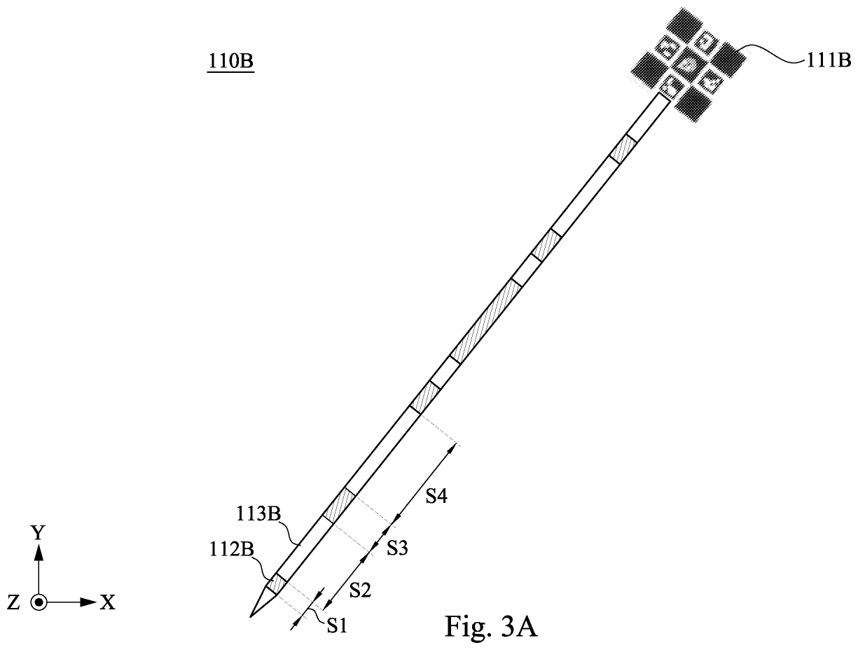

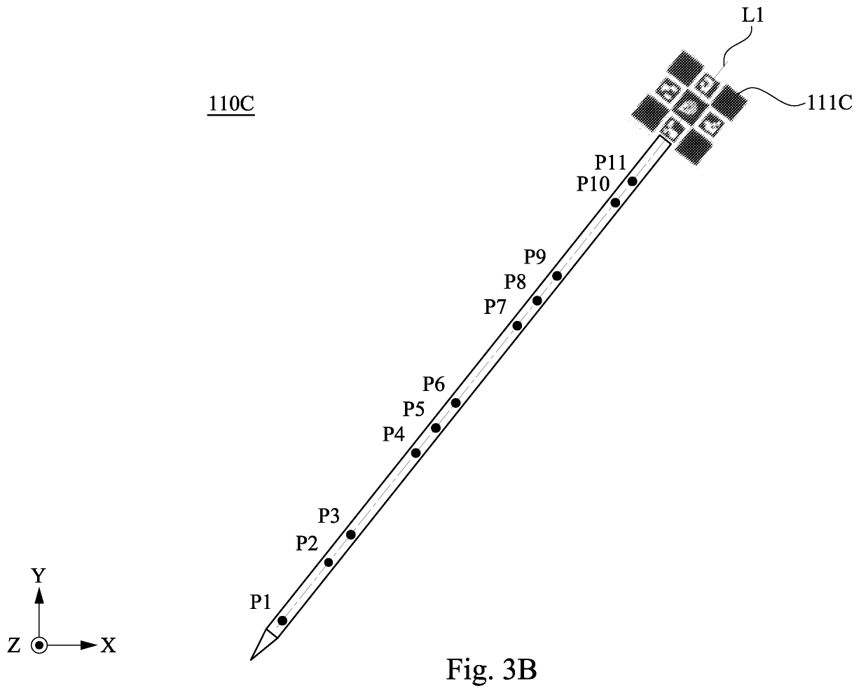

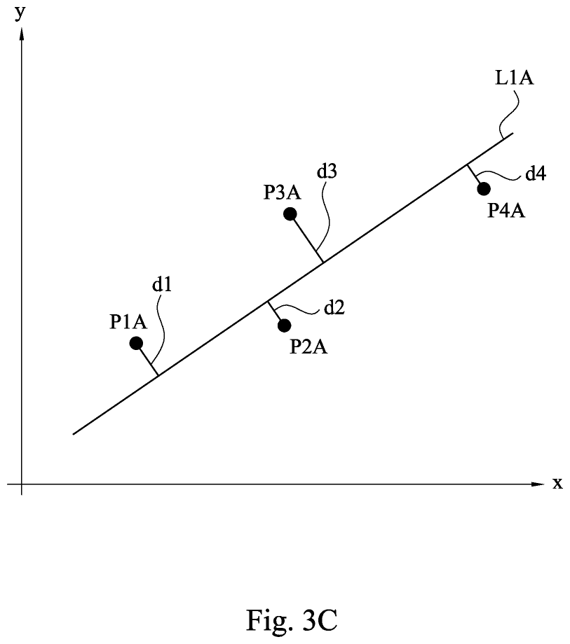

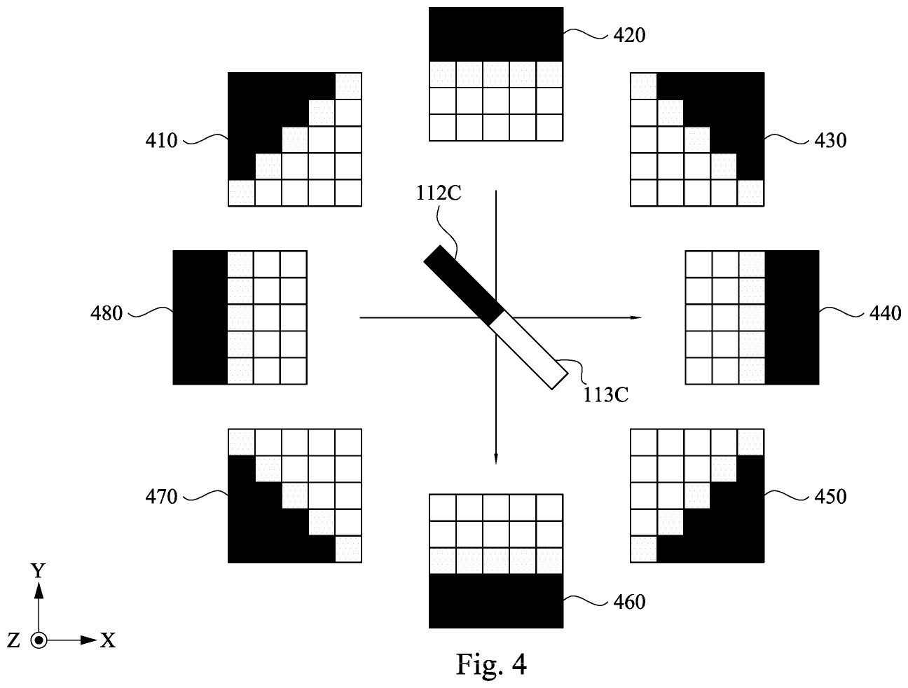

FIG. 3A is a usage scenarios diagram of an augmented reality system according to one embodiment of the present disclosure. FIG. 3B is a usage scenarios diagram of an augmented reality system according to one embodiment of the present disclosure. FIG. 3B is a usage scenarios diagram of an augmented reality system according to one embodiment of the present disclosure. FIG. 3C is a usage scenarios diagram of an augmented reality system according to one embodiment of the present disclosure. FIG. 4 is a usage scenarios diagram of an augmented reality system according to one embodiment of the present disclosure.

In one embodiment, please refer to FIG. 1 to FIG. 4 together, the processor 142 (as shown in FIG. 1) is used to perform following steps according to a plurality commands of the memory 141: capturing the location code 111, the first marking area 112, and the second marking area 113 of the needle 110 to perform positioning or a marking pose estimation by the camera 120.

For example, the processor 142 can be a single processor or an integrated device of multiple microprocessors. Such as a central processing unit (CPU), a graphics processing unit (GPU), or an application-specific integrated circuit (ASIC) . . . etc., but the present disclosure is not limited to this embodiment.

For example, the camera 120 in FIG. 1 can correspond to the camera 120A in FIG. 2, the needle 110 in FIG. 1 can correspond to the needle 110A in FIG. 2, the needle 110B in FIG. 3A, and/or the needle 110C in FIG. 3B. The location code 111 in FIG. 1 can correspond to the location code 111A in FIG. 2, the location code 111B in FIG. 3A, and/or the location code 111C in FIG. 3B. The first marking area 112 in FIG. 1 can correspond to the first marking area 112B in FIG. 3A, the second marking area 113 in FIG. 1 can correspond to the second marking area 113B in FIG. 3A, but the present disclosure is not limited to this embodiment.

For example, the camera 120A can capture the location code 111A of the needle 110A to perform two-dimensional or three-dimensional position conversion, and project the spatial relationship between the needle 110A and the measuring object 90 onto the display. Furthermore, the camera 120A continues to capture the first marking area 112B and the second marking area 113B of the needle 110B to perform the marking pose estimation, that is, the camera 120A continues to capture the first marking area 112B and the second marking area 113B to locate the position of the needle 110B in the space, but the present disclosure is not limited to this embodiment.

Then, confirming the first marking area 112B and the second marking area 113B of the needle 110B are located on a straight line for a feature pose estimation according to a linear regression algorithm.

For example, there can be a plurality of feature points P1 to P11 (as shown in FIG. 3B) between the plurality of first marking areas 112B and plurality of second marking areas 113B, such as there is a feature point P1 between the first marking area 112B and the second marking area 113B, and the plurality of feature points P1 to P11 can be located on a straight line L1, but the present disclosure is not limited to this embodiment. In some embodiments, the linear regression algorithm can obtain the straight line L1 by the first marking area 112B and the second marking area 113B, but the present disclosure is not limited to this embodiment. In some embodiments, the linear regression algorithm can obtain the straight line L1 by the plurality of feature points P1 to P11, but the present disclosure is not limited to this embodiment.

In addition, the linear regression algorithm can fit the following relation 1.

$$Eodr = \frac{1}{n} distance ( Pi , Lodr ) … \qquad relation 1.$$

In some embodiments, the linear regression algorithm can be an algorithm based on the relation 1. In the relation 1, a coordinate Pi can be a plurality of feature points P1 to P11 (as shown in FIG. 3B), a line segment Lodr can be a straight line L1, a parameter n can be a positive integer greater than 0, a parameter Eodr can be an error value, but the present disclosure is not limited to this embodiment. In some embodiments, the line segment Lodr can be an orthogonal distance regression line, but the present disclosure is not limited to this embodiment.

In some embodiments, the meaning of the error Eodr in the relation 1 is averaging the n projection distances (such as a plurality of distances d1 to d4) in FIG. 3C, and the plurality of distances d1 to d4 can be the distance from the feature points P1A to P4A projected to the straight line L1A, but the present disclosure is not limited to this embodiment.

In some embodiments, the number of detectable feature points P1 to P11 of the needle 110A, 110B, or 110C before and after being punctured into the body (such as measuring object 90) is different. The processor 142 takes the average of the distance errors so that the error Eodr has a consistent evaluation standard. When the error Eodr is smaller, it means that the needle 110A, 110B, or 110C is closer to the straight line L1. It is worth noting that most applications of the regression analysis use the ordinary least squares method, the error is calculated as the vertical axis distance, and only the y axis results are considered. However, in the image coordinates, the x axis and y axis are equally important. Therefore, the processor 142 uses the orthogonal distance as the error calculation. Otherwise, for nearly vertical line segments, the error calculated by the vertical distance will be seriously underestimated, but the present disclosure is not limited to this embodiment.

Afterwards, comparing the first marking area 112C (as shown in FIG. 4), the second marking area 113C, and a matching template 410 to 480 is performed to confirm a direction of the needle.

For example, the first marking area 112C and the second marking area 113C in FIG. 4 can respectively correspond to the first marking area 112B and the second marking area 113B in FIG. 3A, and the needle in FIG. 4 can correspond to the needle 110B in FIG. 3A. The memory 141 can store the matching template 410 to 480, the matching template 410 to 480 can correspond to the different directions of the needle 110B, but the present disclosure is not limited to this embodiment. In some embodiments, the first marking area 112C and the second marking area 113C can correspond to the matching template 410, at this time, the tip of the needle can be facing the upper left or lower right, but the present disclosure is not limited to this embodiment.

In some embodiments, the matching template 410 to 480 can correspond to the arranging direction of the first marking area 112B and the second marking area 113B in different directions, so achieve the effect of quickly confirming the different directions of needle 110B. The processor 142 can continuously capture the first marking area 112B and the second marking area 113B on the needle 110B through the needle 110B, so the processor 142 will not misjudge the needle tip position of the needle 110B, but the present disclosure is not limited to this embodiment.

Then, obtaining a feature points number according to the first marking area 112B and the second marking area 113B is performed, and the feature points number is a positive integer greater than or equal to 2.

For example, the processor 142 can obtain the plurality of feature points P1 to P11 in FIG. 3B through the plurality of first marking area 112B and the plurality of second marking area 113B in FIG. 3A, and a number of the plurality of feature points P1 to P11 can be 11, but the present disclosure is not limited to this embodiment. In some embodiments, the plurality of first marking area 112B and the plurality of second marking area 113B in FIG. 3A can truly exist in the needle 110B, the plurality of feature points P1 to P11 only are the feature points displayed (or annotated) by the processor 142 on a display screen, and the above feature points do not truly exist on the needle 110B or 110C, but the present disclosure is not limited to this embodiment.

Afterwards, obtaining a range prediction interval of the ultrasound field of view 200 is performed according to the feature points number of the needle 110C.

For example, the processor 142 can project the needle 110C into the ultrasound field of view 200 according to the feature points number of the needle 110C. The projection of the needle 110C can be the predicted projection range size, and the above predicted projection range size is the range prediction interval, but the present disclosure is not limited to this embodiment.

Then, increasing or decreasing the range prediction interval is performed according to the feature points number and a feature points number threshold.

For example, the feature points number threshold can be set according to user needs, the processor 142 can increase or decrease the range prediction interval according to the feature points number is greater than or smaller than the feature points number threshold, but the present disclosure is not limited to this embodiment.

Afterwards, when it is determined that the feature points number is greater than the feature points number threshold, the range prediction interval is reduced.

For example, when the feature points number is greater than the feature points number threshold, the range prediction interval can be reduced to increase a confidence of the range prediction interval of the needle 110C projected to the ultrasound field of view 200. That is, the smaller the range prediction interval, the greater the confidence, but the present disclosure is not limited to this embodiment.

In some embodiments, the processor 142 (as shown in FIG. 1) is further used to perform the following steps according to the plurality commands of the memory 141: confirming the first marking area 112B and the second marking area 113B of the needle are located on the straight line for a needle bending detection according to at least one of the linear regression algorithm and a bending relational expression, wherein the bending relational expression has a distance error mean value.

For example, the bending relational expression can be the above relation 1, the distance error mean value can be the above parameter Eodr, the straight line can be the straight line L1 of FIG. 3B or the straight line L1A of FIG. 3C, but the present disclosure is not limited to this embodiment. In addition, the needle bending detection can detect whether the needle 110B has exceeded the tolerable curvature. When the needle 110B is too curved, it may easily cause the augmented reality system 100 of the present disclosure to fail (such as positioning misalignment of the needle 110B). The present disclosure can continuously detect the straight line (or the curvature) of the needle 110B to achieve the effect of avoid positioning failure, but the present disclosure is not limited to this embodiment.

Then, setting a bending threshold is performed according to the bending relational expression.

For example, the bending threshold can be 1.5 Pixel, but the present disclosure is not limited to this embodiment. In addition, the bending threshold can be related to the length of the needle 110B, but the present disclosure is not limited to this embodiment.

Afterwards, when the distance error mean value smaller than the bending threshold, it is determined that the needle 110B approaches the straight line.

For example, when the parameter Eodr is stably maintained below 1.5 pixel, the processor 142 can determine that the needle 110B is approaching the straight line, but the present disclosure is not limited to this embodiment.

In some embodiments, please refer to FIG. 3A, a length S1 of the first marking area 112B is smaller than a length S2 of the second marking area 113B.

For example, the plurality of first marking area 112B and the plurality of second marking area 113B of the needle 110B can be staggered with irregular lengths, and the length S1 of the first marking area 112B can be greater than or smaller than the length S2 of the second marking area 113B, but the present disclosure is not limited to this embodiment.

In some embodiments, the plurality of first marking area 112B have the length S1 and a length S3, the plurality of second marking area 113B can have the length S2 and a length S4, and plurality of length S1˜S4 can different from each other, but the present disclosure is not limited to this embodiment.

In some embodiments, the length S1 of the first marking area 112B is equal to the length S2 of the second marking area 113B.

For example, the plurality of first marking area 112B and the plurality of second marking area 113B of the needle 110B can be staggered with regular lengths, and the length S1 of the first marking area 112B can be equal to the length S2 of the second marking area 113B, but the present disclosure is not limited to this embodiment. In some embodiments, the plurality of lengths S1 to S4 can be the same from each other, but the present disclosure is not limited to this embodiment.

In some embodiments, a distance between the feature point P1 and the feature point P2 in FIG. 3B can be the length S2 in FIG. 3A, the distances of other feature points can be deduced by analogy, but the present disclosure is not limited to this embodiment.

In some embodiments, the camera 120A in FIG. 2 can capture the plurality of first marking area 112B and the plurality of second marking area 113B on the needle 110A, 110B as much as possible according to the lens focal length, the depth of field, the resolution, the field of view, and other hardware limitations of the camera 120A. The processor 142 obtains the plurality of feature points P1 to P11. It should be noted that when the processor 142 indeed captures the second marking area 113B adjacent to the first marking area 112B, the augmented reality system 100 can perform the technology described in the present disclosure (such as performing positioning, the marking pose estimation, or the feature pose estimation), but the present disclosure is not limited to this embodiment.

In some embodiments, when the processor 142 indeed obtains at least two of the plurality of feature points P1 to P11, the augmented reality system 100 can execute the technology described in the present disclosure, but the present disclosure is not limited to this embodiment.

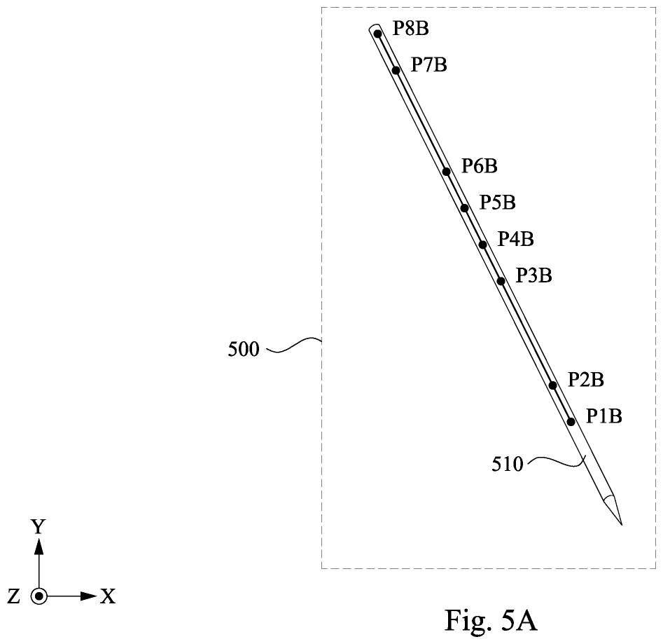

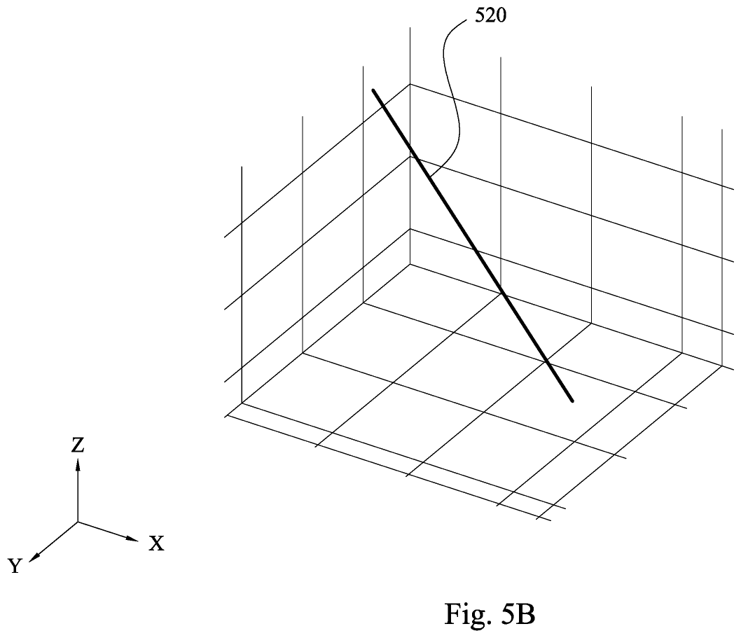

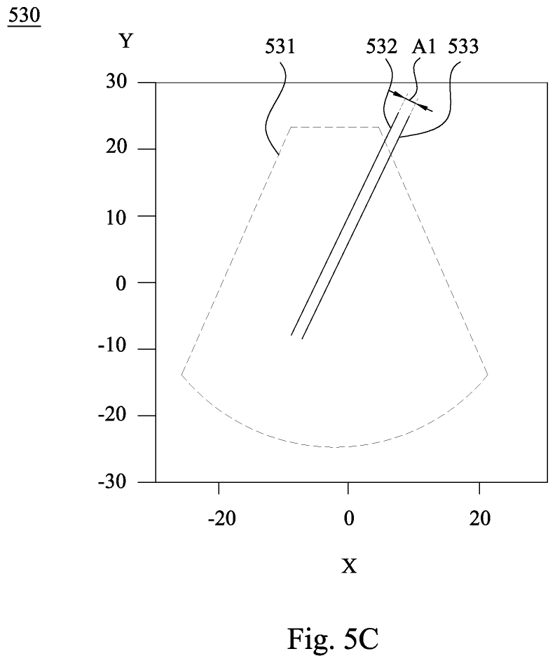

FIGS. 5A to 5C are usage scenarios diagrams of an augmented reality system according to one embodiment of the present disclosure. As shown in FIG. 5A, in some embodiments, a needle 510 in FIG. 5A can correspond to the needle 110B in FIG. 3A, a picture 500 can be the picture captured by the camera 120A (as shown in FIG. 2) for the needle 110B in FIG. 3A. The plurality of feature points P1B to P8B in FIG. 5A can correspond to the plurality of feature points P1 to P11 in FIG. 3B, and the feature points number of the plurality of feature points P1B to P8B can be 8, but the present disclosure is not limited to this embodiment.

As shown in FIG. 5B, in some embodiments, a line segment 520 can be a projection line segment of the needle 510 of FIG. 5A projected to three-dimensional coordinates, but the present disclosure is not limited to this embodiment.

As shown in FIG. 5C, in some embodiments, the ultrasound field of view 531 can correspond to the ultrasound field of view 200 of FIG. 2, the line segment 532 and the line segment 533 can form the range prediction interval, and the range prediction interval have a width A1, but the present disclosure is not limited to this embodiment. In some embodiments, the width A1 can be related to the feature points number of the plurality of feature points P1B to P8B, but the present disclosure is not limited to this embodiment.

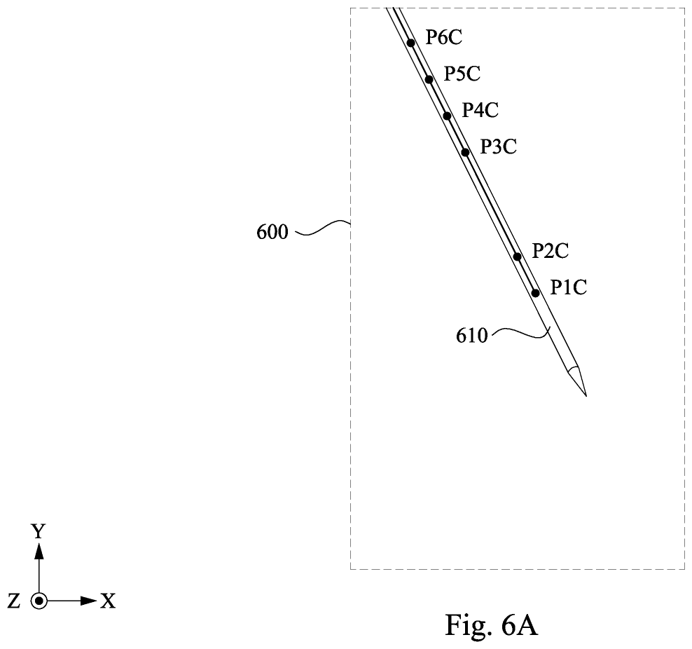

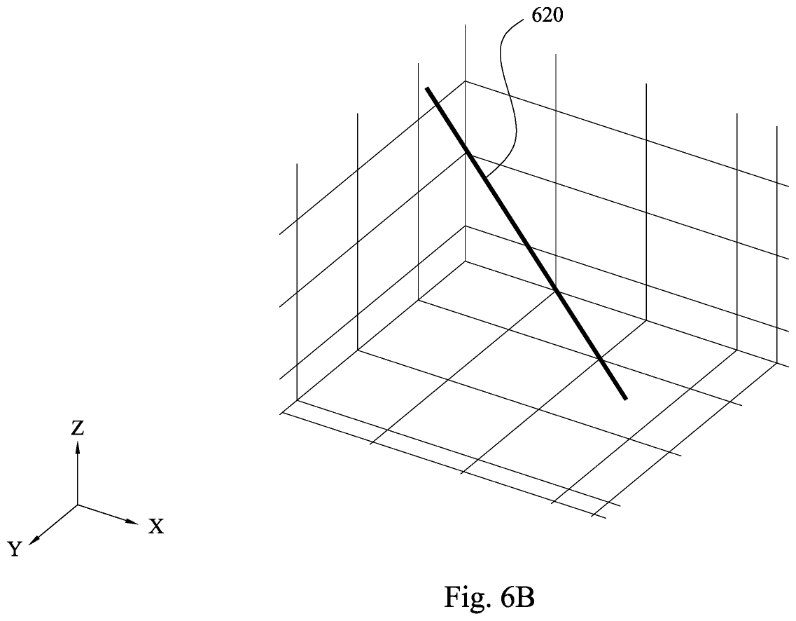

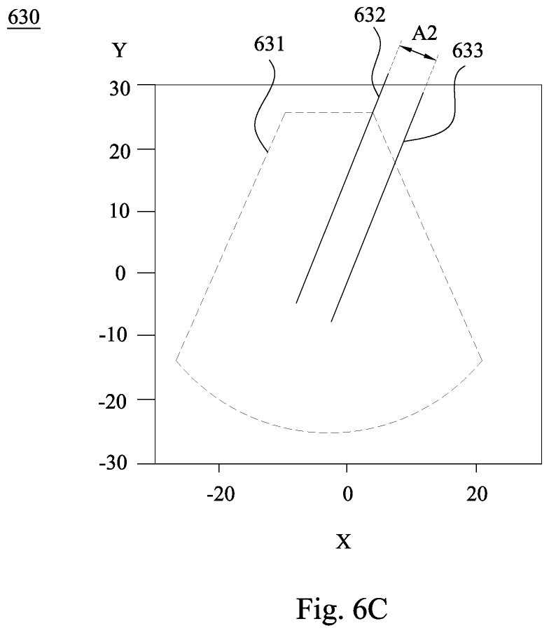

FIGS. 6A to 6C are usage scenarios diagrams of an augmented reality system according to one embodiment of the present disclosure. As shown in FIG. 6A, the needle 610 of the picture 600 can be a moved picture of the needle 510 of the picture 500, and the plurality of feature points P1C to P6C can correspond to the plurality of feature points P1B to P6B in FIG. 5A, and the feature points number of the plurality of feature points P1C to P6C can be 6, but the present disclosure is not limited to this embodiment.

As shown in FIG. 6B, in some embodiments, the line segment 620 can be the projection line segment of the needle 610 of FIG. 6A projected to three-dimensional coordinates, but the present disclosure is not limited to this embodiment. in some embodiments, a width of the line segment 620 in FIG. 6B can be greater than a width of the line segment 520 in FIG. 5B, but the present disclosure is not limited to this embodiment.

As shown in FIG. 6C, in some embodiments, the ultrasound field of view 631 can correspond to the ultrasound field of view 200 in FIG. 2, the line segment 632 and the line segment 633 can form the range prediction interval, and the range prediction interval have the width A2, but the present disclosure is not limited to this embodiment. In some embodiments, the width A2 can be related to the feature points number of the plurality of feature points P1C to P6C, but the present disclosure is not limited to this embodiment. In some embodiments, the width A2 of FIG. 6C can be greater than the width A1 of FIG. 5C, that is, the confidence of the range prediction interval in FIG. 6C is less than the confidence of the range prediction interval in FIG. 5C, but the present disclosure is not limited to this embodiment.

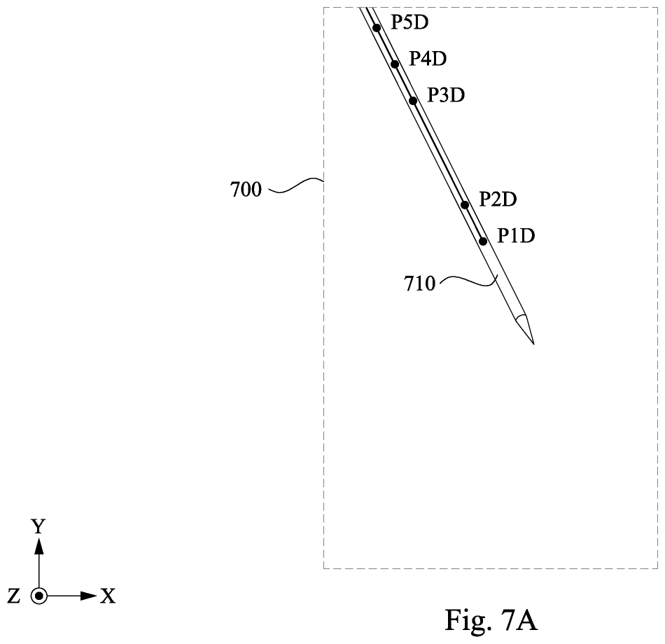

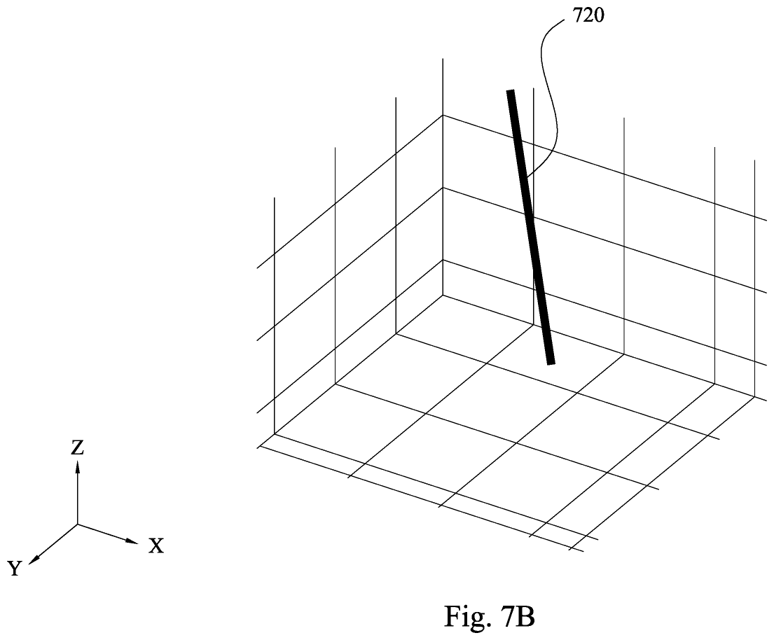

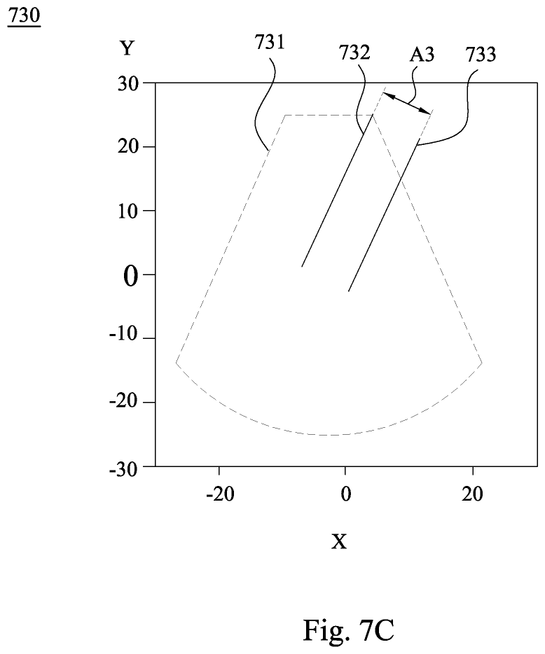

FIGS. 7A to 7C are usage scenarios diagrams of an augmented reality system according to one embodiment of the present disclosure. As shown in FIG. 7A, 6A, the needle 710 of the picture 700 can be a moved picture of the needle 610 of the picture 600, and the plurality of feature points P1D to P5D can correspond to the plurality of feature points P1C to P5C in FIG. 6A, and the feature points number of the plurality of feature points P1D to P5D can be 5, but the present disclosure is not limited to this embodiment.

As shown in FIG. 7B, in some embodiments, he line segment 720 can be the projection line segment of the needle 710 of FIG. 7A projected to three-dimensional coordinates, but the present disclosure is not limited to this embodiment. In some embodiments, the width of the line segment 720 in FIG. 7B can be greater than the width of the line segment 620 in FIG. 6B, but the present disclosure is not limited to this embodiment.

As shown in FIG. 7C, in some embodiments, the ultrasound field of view 731 can correspond to the ultrasound field of view 200 in FIG. 2, the line segment 732 and line segment 733 can form the range prediction interval, and the range prediction interval have width A3, but the present disclosure is not limited to this embodiment. In some embodiments, the width A3 can be related to the feature points number of the feature points P1D to P5D, but the present disclosure is not limited to this embodiment. In some embodiments, the width A3 of FIG. 7C can be greater than the width A2 of FIG. 6C, that is, the confidence of the range prediction interval in FIG. 7C is less than the confidence of the range prediction interval in FIG. 6C, but the present disclosure is not limited to this embodiment.

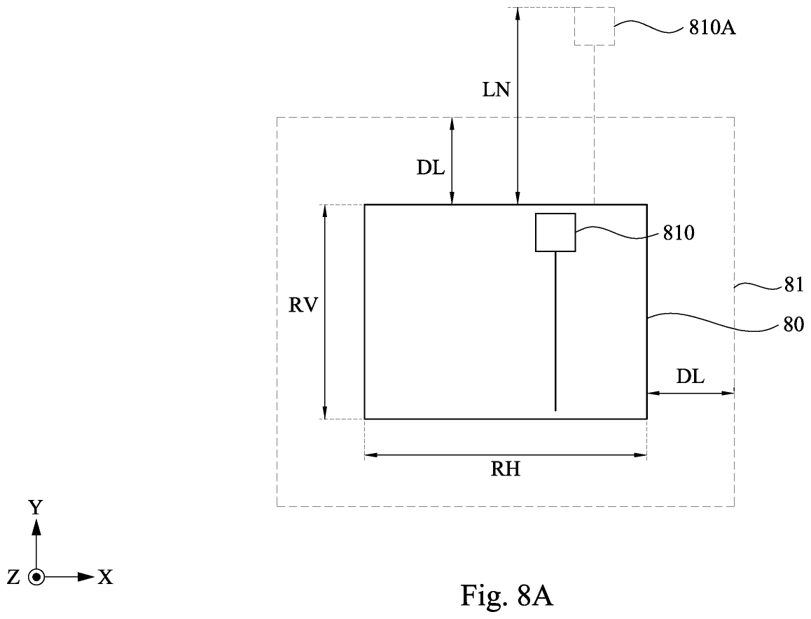

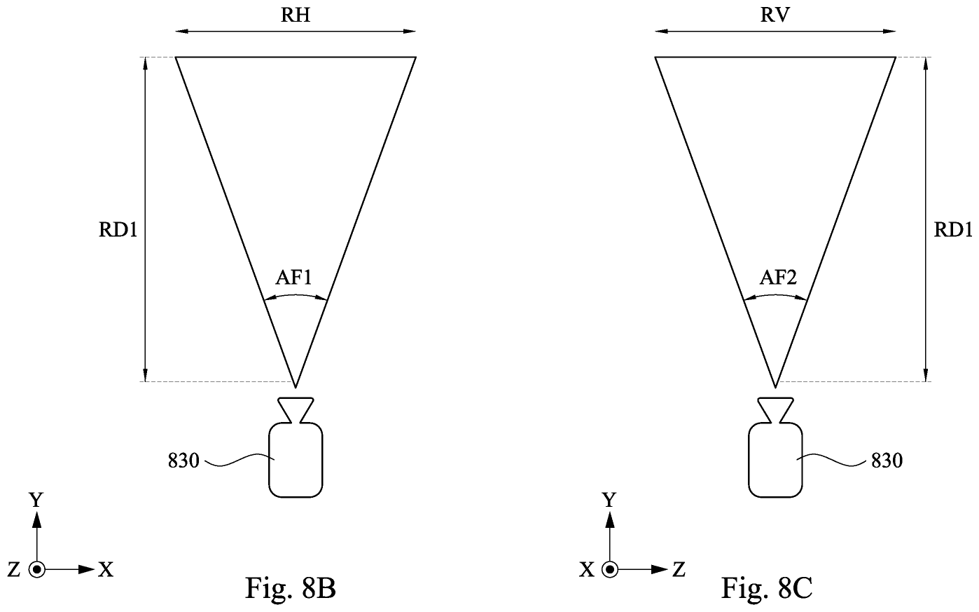

FIGS. 8A to 8C are usage scenarios diagrams of an augmented reality system according to one embodiment of the present disclosure. As shown in FIGS. 8A to 8C, in some embodiments, the camera 830 can obtain the field of view 80 of the needle 810 through the depth of field RD1, the field of view 80 can be expanded to an expanded field of view through the technology of the present disclosure (such as performing positioning, the marking pose estimation and/or the feature pose estimation), and the needle 810 can be moved to the position of the needle 810A at will, but the present disclosure is not limited to this embodiment.

In some embodiments, the camera 830 of FIG. 8B and FIG. 8C can correspond to the camera 120 of FIG. 1 and/or the camera 120A of FIG. 2. The needle 810, 810A of FIG. 8A can correspond to the needle 110 of FIG. 1, the needle 110A of FIG. 2, the needle 110B of FIG. 3A, the needle 110C of FIG. 3B, the needle 510 of FIG. 5A, the needle 610 of FIG. 6A, and/or the needle 710 of FIG. 7A, but the present disclosure is not limited to this embodiment.

In some embodiments, the processor 142 (as shown in FIG. 1) is further used to perform the following steps according to the plurality commands of the memory 141: obtaining a depth of field length RD1 between the camera 830 and the measuring object (such as the needle 810); and obtaining a horizontal image range RH according to a horizontal viewing angle AF1 and the depth of field length RD1 of the camera 830.

For example, the horizontal image range RH can fit the following relation 2, the depth of field length RD1 can be 50 centimeter (cm), and the horizontal viewing angle AF1 can be 0 to 90 degrees. The horizontal image range RH can be 30.2 centimeter, but the present disclosure is not limited to this embodiment.

$$RH = 2 \times D L \times tan ( \frac{AF 1}{2} ) … \qquad relation 2.$$

In some embodiments, the processor 142 (as shown in FIG. 1) is further used to perform the following steps according to the plurality commands of the memory 141: obtaining a vertical image range RV according to a vertical viewing angle AF2 and the depth of field length RD1 of the camera 830.

For example, the vertical image range RV can fit the following relation 3, the vertical viewing angle AF2 can be 0 to 90 degrees, the vertical image range RV can be 22.7 centimeter, but the present disclosure is not limited to this embodiment.

$$RV = 2 \times DL \times tan ( \frac{AF 2}{2} ) … \qquad relation 3.$$

In some embodiments, the processor 142 (as shown in FIG. 1) is further used to perform the following steps according to the plurality commands of the memory 141: obtaining a horizontal elongation according to the horizontal image range RH and an effective length DL of the needle 810A.

For example, the effective length DL of the needle 810A can be the maximum distance that the needle 810A can be lifted from the boundary of the field of view 80 before losing tracking (or capturing). The effective length DL of the needle 810A can fit the following relation 4, the horizontal elongation can fit the following relation 5, and the horizontal elongation can be 66%, but the present disclosure is not limited to this embodiment.

$$D L = L N - L V … \qquad relation 4.$$

In some embodiments, in the relation 4, the parameter LN can be a total length of the needle 810A, the parameter LV can be the needle length remaining in the field of view 80, the parameter DL can be an effective length of the needle 810A, but the present disclosure is not limited to this embodiment.

$$IH = \frac{2 \times DL}{RH} … \qquad relation 5.$$

In some embodiments, in the relation 5, the parameter IH can be the horizontal elongation, the parameter DL can be the effective length of the needle 810A, and the parameter RH can be the horizontal image range, but the present disclosure is not limited to this embodiment.

In some embodiments, the effective length DL is greater than or equal to a sum of the first length S1 of the first marking area 112B and the second length S2 of the second marking area 113B.

For example, the effective length DL can be greater than or equal to a sum of the first length S1 and the second length S2, but the present disclosure is not limited to this embodiment.

In some embodiments, the processor 142 (as shown in FIG. 1) is further used to perform the following steps according to the plurality commands of the memory 141: obtaining a vertical elongation according to the vertical image range RV and the effective length of the needle 810A.

For example, the vertical elongation can fit the following relation 6, the vertical elongation can be 88%, but the present disclosure is not limited to this embodiment.

$$IV = \frac{2 \times DL}{RV} … \qquad relation 6.$$

In some embodiments, in the relation 6, the parameter IV can be the vertical elongation, the parameter DL can be the effective length of the needle 810A, and the parameter RV can be the vertical image range, but the present disclosure is not limited to this embodiment.

In some embodiments, the processor 142 (as shown in FIG. 1) is further used to perform the following steps according to the plurality commands of the memory 141: obtaining an area elongation according to the horizontal image range RH, the vertical image range RV, and the effective length DL of the needle 810A.

For example, the area elongation can fit the following relation 7, the area elongation can be 213%, but the present disclosure is not limited to this embodiment.

$$IA = \frac{( RH \times 2 \times DL ) \times ( RV + 2 DL )}{RH \times RV} - 1 … \qquad relation 7.$$

In some embodiments, in the relation 6, the parameter IA can be the area elongation, the parameter RH can be the horizontal image range, the parameter RV can be the vertical image range, and the parameter DL can be the effective length of the needle 810A, but the present disclosure is not limited to this embodiment.

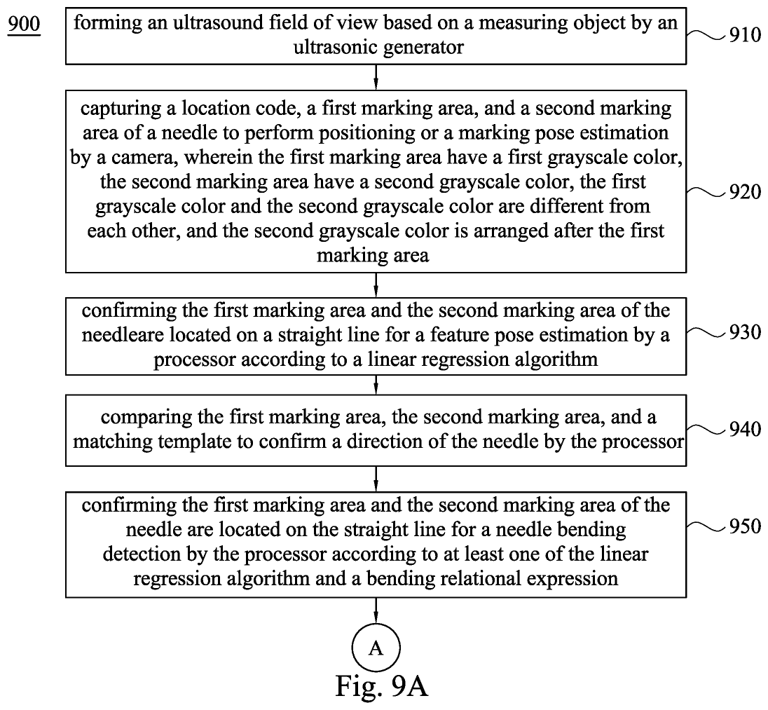

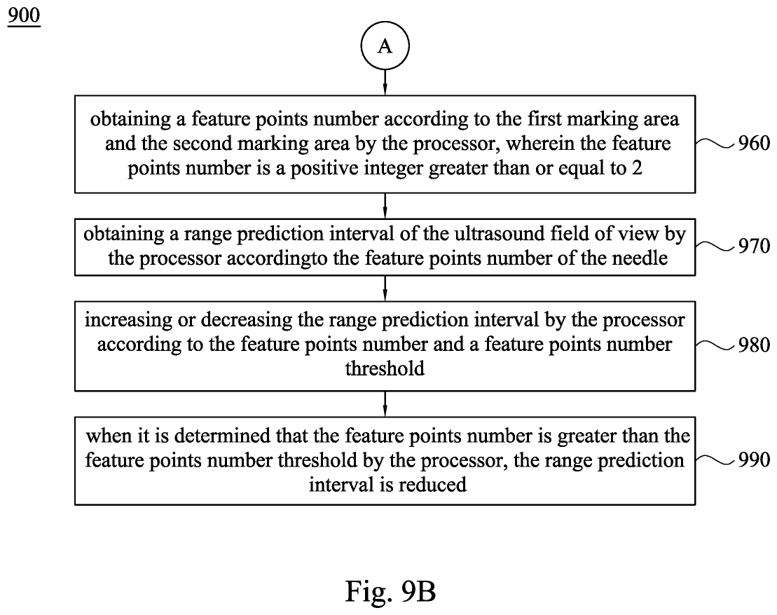

FIGS. 9A to 9B are step flowing diagrams of an augmented reality method according to one embodiment of the present disclosure. As shown in FIG. 9A to 9B, the augmented reality method 900 includes a plurality of steps 910 to 990, the steps of the augmented reality method 900 in FIG. 9A to 9B are detailed below.

In step 910, forming an ultrasound field of view based on a measuring object by an ultrasound generator.

In one embodiment, please refer to FIG. 1, FIG. 2, and FIG. 9A together, the ultrasound generator 130 or 130A can be used to form the ultrasound field of view 200 based on the measuring object 90. For example, the operations of the augmented reality method 900 are similar to the operations of the augmented reality system 100 in FIG. 1 and/or FIG. 2, and the descriptions regarding the other operations of the augmented reality method 900 will be omitted herein for the sake of brevity.

In step 920, capturing a location code, a first marking area, and a second marking area of a needle to perform positioning or a marking pose estimation by a camera, the first marking area has a first grayscale color, the second marking area has a second grayscale color, the first grayscale color and the second grayscale color are different from each other, and the second grayscale color is arranged after the first marking area.

In one embodiment, please refer to FIG. 1, FIG. 2, and FIG. 9A together, the location code 111, the first marking area 112, and the second marking area 113 of the needle 110 can be captured by the camera 120 to perform positioning or a marking pose estimation. For example, the operations of the augmented reality method 900 are similar to the operations of the augmented reality system 100 in FIG. 1 and/or FIG. 2, and the descriptions regarding the other operations of the augmented reality method 900 will be omitted herein for the sake of brevity.

In step 930, confirming the first marking area and the second marking area of the needle are located on a straight line for a feature pose estimation by a processor according to a linear regression algorithm.

In one embodiment, please refer to FIG. 1, FIG. 3A, and FIG. 9A together, the processor 142 can confirm that the first marking area 112B and the second marking area 113B of the needle 110B are located on the straight line for the feature pose estimation according to the linear regression algorithm. For example, the operations of the augmented reality method 900 are similar to the operations of the augmented reality system 100 in FIG. 1 and/or FIG. 3A, and the descriptions regarding the other operations of the augmented reality method 900 will be omitted herein for the sake of brevity.

In step 940, comparing the first marking area, the second marking area, and a matching template to confirm a direction of the needle by the processor.

In one embodiment, please refer to FIG. 1, FIG. 4, and FIG. 9A together, the processor 142 can compare the first marking area 112C, the second marking area 113C, and the matching template 410 to 480 to confirm the direction of the needle. For example, the operations of the augmented reality method 900 are similar to the operations of the augmented reality system 100 in FIG. 1 and/or FIG. 4, and the descriptions regarding the other operations of the augmented reality method 900 will be omitted herein for the sake of brevity.

In step 950, confirming the first marking area and the second marking area of the needle are located on the straight line for a needle bending detection by the processor according to at least one of the linear regression algorithm and a bending relational expression.

In one embodiment, please refer to FIG. 1, FIG. 3A, and FIG. 9A together, the processor 142 can confirm the first marking area 112B and the second marking area 113B of the needle are located on the straight line for the needle bending detection according to at least one of the linear regression algorithm and the bending relational expression. For example, the operations of the augmented reality method 900 are similar to the operations of the augmented reality system 100 in FIG. 1 and/or FIG. 3A, and the descriptions regarding the other operations of the augmented reality method 900 will be omitted herein for the sake of brevity.

In step 960, obtaining a feature points number according to the first marking area and the second marking area by the processor, wherein the feature points number is a positive integer greater than or equal to 2.

In one embodiment, please refer to FIG. 1, FIG. 3A, and FIG. 9B together, the processor 142 can obtain the feature points number according to the first marking area 112B and the second marking area 113B, and the feature points number is the positive integer greater than or equal to 2. For example, the operations of the augmented reality method 900 are similar to the operations of the augmented reality system 100 in FIG. 1 and/or FIG. 3A, and the descriptions regarding the other operations of the augmented reality method 900 will be omitted herein for the sake of brevity.

In step 970, obtaining a range prediction interval of the ultrasound field of view by the processor according to the feature points number of the needle.

In one embodiment, please refer to FIG. 1, FIG. 2, FIG. 3B, and FIG. 9B together, the processor 142 can obtain the range prediction interval of the ultrasound field of view 200 according to the feature points number of the needle 110C. For example, the operations of the augmented reality method 900 are similar to the operations of the augmented reality system 100 in FIG. 1, FIG. 2 and/or FIG. 3A, and the descriptions regarding the other operations of the augmented reality method 900 will be omitted herein for the sake of brevity.

In step 980, increasing or decreasing the range prediction interval by the processor according to the feature points number and a feature points number threshold.

In one embodiment, please refer to FIG. 1, FIG. 3B, and FIG. 9B together, the processor 142 can increase or decrease the range prediction interval according to the feature points number (such as the number of the plurality of feature points P1 to P11) and a feature points number threshold. For example, the operations of the augmented reality method 900 are similar to the operations of the augmented reality system 100 in FIG. 1 and/or FIG. 3B, and the descriptions regarding the other operations of the augmented reality method 900 will be omitted herein for the sake of brevity.

In step 990, when it is determined that the feature points number is greater than the feature points number threshold by the processor, the range prediction interval is reduced.

In one embodiment, please refer to FIG. 1, FIG. 3B, and FIG. 9B together, when the processor 142 determines that the feature points number (such as the number of the plurality of feature points P1 to P11) is greater than the feature points number threshold by the processor, the range prediction interval is reduced. For example, the operations of the augmented reality method 900 are similar to the operations of the augmented reality system 100 in FIG. 1 and/or FIG. 3B, and the descriptions regarding the other operations of the augmented reality method 900 will be omitted herein for the sake of brevity.

In one embodiment, please refer to FIGS. 3A, 9A, and 9B together, the length S1 of the first marking area 112B is smaller than the length S2 of the second marking area 113B. For example, the operations of the augmented reality method 900 are similar to the operations of the augmented reality system 100 in FIG. 3A, and the descriptions regarding the other operations of the augmented reality method 900 will be omitted herein for the sake of brevity.

In one embodiment, please refer to FIG. 3A, FIG. 9A, and FIG. 9B together, the length S1 of the first marking area 112B is equal to the length S2 of the second marking area 113B. For example, the operations of the augmented reality method 900 are similar to the operations of the augmented reality system 100 in FIG. 3A, and the descriptions regarding the other operations of the augmented reality method 900 will be omitted herein for the sake of brevity.

In one embodiment, please refer to FIG. 1, FIG. 3B, FIG. 9A, and FIG. 9B together, the augmented reality method 900 further includes the following steps: setting the bending threshold by the processor 142 according to the bending relational expression, and the bending relational expression has the distance error mean value; and when the distance error mean value smaller than the bending threshold, it is determined that the needle 110C approaches the straight line L1 by the processor 142. For example, the operations of the augmented reality method 900 are similar to the operations of the augmented reality system 100 in FIG. 1 and/or FIG. 3B, and the descriptions regarding the other operations of the augmented reality method 900 will be omitted herein for the sake of brevity.

In one embodiment, please refer to FIG. 1, FIG. 8A to 8C, FIG. 9A, and FIG. 9B together, the augmented reality method 900 further includes the following steps: obtaining the depth of field length RD1 between the camera 830 and the measuring object (such as the needle 810) by the processor 142; and obtaining the horizontal image range by the processor 142 according to the horizontal viewing angle AF1 and the depth of field length RD1 of the camera 830. For example, the operations of the augmented reality method 900 are similar to the operations of the augmented reality system 100 in FIG. 1 and/or FIG. 8A to 8C, and the descriptions regarding the other operations of the augmented reality method 900 will be omitted herein for the sake of brevity.

In one embodiment, please refer to FIG. 1, FIG. 8A to 8C, FIG. 9A, and FIG. 9B together, the augmented reality method 900 further includes the following steps: obtaining the vertical image range RV by the processor 142 according to a vertical viewing angle AF2 and the depth of field length RD1 of the camera 830. For example, the operations of the augmented reality method 900 are similar to the operations of the augmented reality system 100 in FIG. 1 and/or FIG. 8A to 8C, and the descriptions regarding the other operations of the augmented reality method 900 will be omitted herein for the sake of brevity.

In one embodiment, please refer to FIG. 1, FIG. 8A to 8C, FIG. 9A, and FIG. 9B together, the augmented reality method 900 further includes the following steps: obtaining the horizontal elongation by the processor 142 according to the horizontal image range RH and the effective length DL of the needle 810A. For example, the operations of the augmented reality method 900 are similar to the operations of the augmented reality system 100 in FIG. 1 and/or FIG. 8A to 8C, and the descriptions regarding the other operations of the augmented reality method 900 will be omitted herein for the sake of brevity.

In one embodiment, please refer to FIG. 1, FIG. 8A to 8C, FIG. 9A, and FIG. 9B together, the effective length DL is greater than or equal to the sum of the first length S1 of the first marking area 112B and the second length S2 of the second marking area 113B. For example, the operations of the augmented reality method 900 are similar to the operations of the augmented reality system 100 in FIG. 1 and/or FIG. 8A to 8C, and the descriptions regarding the other operations of the augmented reality method 900 will be omitted herein for the sake of brevity.

In one embodiment, please refer to FIG. 1, FIG. 8A to 8C, FIG. 9A, and FIG. 9B together, the augmented reality method 900 further includes the following steps: obtaining the vertical elongation by the processor 142 according to the vertical image range RV and the effective length DL of the needle 810A. For example, the operations of the augmented reality method 900 are similar to the operations of the augmented reality system 100 in FIG. 1 and/or FIG. 8A to 8C, and the descriptions regarding the other operations of the augmented reality method 900 will be omitted herein for the sake of brevity.

In one embodiment, please refer to FIG. 1, FIG. 8A to 8C, FIG. 9A, and FIG. 9B together, the augmented reality method 900 further includes the following steps: obtaining the area elongation by the processor 142 according to the horizontal image range RH, the vertical image range RV, and the effective length DL of the needle 810A. For example, the operations of the augmented reality method 900 are similar to the operations of the augmented reality system 100 in FIG. 1 and/or FIG. 8A to 8C, and the descriptions regarding the other operations of the augmented reality method 900 will be omitted herein for the sake of brevity.

In some embodiments, the step A in FIG. 9A and the step A in FIG. 9B can be the same step. For example, the step A can be any step or method related to the augmented reality, or the step A can be any step or method used to succeed the step 950 of FIG. 9A and the step 960 of FIG. 9B, but the present disclosure is not limited to this embodiment. In some embodiments, the step A in FIG. 9A and the step A in FIG. 9B can be a node for inheritance, the step 960 can directly follow the step 950 in the augmented reality method 900, but the present disclosure is not limited to this embodiment.

It can be seen from the above embodiments of the present disclosure that the application of the present disclosure has the following advantages. The augmented reality system and the augmented reality method shown in the embodiment of the present disclosure can achieve the effect of expanding the field off view by detecting (or capturing) a plurality of marking areas and a plurality of feature points on the needle.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An augmented reality system, comprising:
a needle, comprising:
a location code;
a first marking area, wherein the first marking area has a first grayscale color; and
a second marking area, wherein the second marking area has a second grayscale color, the first grayscale color and the second grayscale color are different from each other, and the second grayscale color is arranged after the first marking area;
a camera;
an ultrasound generator, configured to form an ultrasound field of view based on a measuring object;
a memory, configured to store a plurality of commands;
a processor, configured to perform following steps according to the plurality of commands of the memory:
capturing the location code, the first marking area, and the second marking area of the needle to perform positioning or a marking pose estimation by the camera;
confirming the first marking area and the second marking area of the needle are located on a straight line for a feature pose estimation according to a linear regression algorithm;
comparing the first marking area, the second marking area, and a matching template to confirm a direction of the needle;
obtaining a feature points number according to the first marking area and the second marking area, wherein the feature points number is a positive integer greater than or equal to 2;
obtaining a range prediction interval of the ultrasound field of view according to the feature points number of the needle;
increasing or decreasing the range prediction interval according to the feature points number and a feature points number threshold; and
when it is determined that the feature points number is greater than the feature points number threshold, the range prediction interval is reduced.

2. The augmented reality system of claim 1, wherein
a length of the first marking area is smaller than a length of the second marking area.

3. The augmented reality system of claim 1, wherein
a length of the first marking area is equal to a length of the second marking area.

4. The augmented reality system of claim 1, wherein
the processor is further configured to perform the following steps according to the plurality of commands of the memory:
confirming the first marking area and the second marking area of the needle are located on the straight line for a needle bending detection according to at least one of the linear regression algorithm and a bending relational expression, wherein the bending relational expression has a distance error mean value;
setting a bending threshold according to the bending relational expression; and
when the distance error mean value smaller than the bending threshold, it is determined that the needle approaches the straight line.

5. The augmented reality system of claim 1, wherein
the processor is further configured to perform the following steps according to the plurality of commands of the memory:
obtaining a depth of field length between the camera and the measuring object; and
obtaining a horizontal image range according to a horizontal viewing angle and the depth of field length of the camera.

6. The augmented reality system of claim 5, wherein
the processor is further configured to perform the following steps according to the plurality of commands of the memory:
obtaining a vertical image range according to a vertical viewing angle and the depth of field length of the camera.

7. The augmented reality system of claim 6, wherein
the processor is further configured to perform the following steps according to the plurality of commands of the memory:
obtaining a horizontal elongation according to the horizontal image range and an effective length of the needle.

8. The augmented reality system of claim 7, wherein
the effective length is greater than or equal to a sum of a first length of the first marking area and a second length of the second marking area.

9. The augmented reality system of claim 7, wherein
the processor is further configured to perform the following steps according to the plurality of commands of the memory:
obtaining a vertical elongation according to the vertical image range and the effective length of the needle.

10. The augmented reality system of claim 9, wherein
the processor is further configured to perform the following steps according to the plurality of commands of the memory:
obtaining an area elongation according to the horizontal image range, the vertical image range, and the effective length of the needle.

11. An augmented reality method, comprising:
forming an ultrasound field of view based on a measuring object by an ultrasound generator;
capturing a location code, a first marking area, and a second marking area of a needle to perform positioning or a marking pose estimation by a camera, wherein the first marking area has a first grayscale color, the second marking area has a second grayscale color, the first grayscale color and the second grayscale color are different from each other, and the second grayscale color is arranged after the first marking area;
confirming the first marking area and the second marking area of the needle are located on a straight line for a feature pose estimation by a processor according to a linear regression algorithm;
comparing the first marking area, the second marking area, and a matching template to confirm a direction of the needle by the processor;
confirming the first marking area and the second marking area of the needle are located on the straight line for a needle bending detection by the processor according to at least one of the linear regression algorithm and a bending relational expression;
obtaining a feature points number according to the first marking area and the second marking area by the processor, wherein the feature points number is a positive integer greater than or equal to 2;
obtaining a range prediction interval of the ultrasound field of view by the processor according to the feature points number of the needle;
increasing or decreasing the range prediction interval by the processor according to the feature points number and a feature points number threshold; and
when it is determined that the feature points number is greater than the feature points number threshold by the processor, the range prediction interval is reduced.

12. The augmented reality method of claim 11, wherein
a length of the first marking area is smaller than a length of the second marking area.

13. The augmented reality method of claim 11, wherein
a length of the first marking area is equal to a length of the second marking area.

14. The augmented reality method of claim 11, further comprising:
setting a bending threshold by the processor according to the bending relational expression, wherein the bending relational expression has a distance error mean value; and
when the distance error mean value smaller than the bending threshold, it is determined that the needle approaches the straight line by the processor.

15. The augmented reality method of claim 11, further comprising:
obtaining a depth of field length between the camera and the measuring object by the processor; and
obtaining a horizontal image range by the processor according to a horizontal viewing angle and the depth of field length of the camera.

16. The augmented reality method of claim 15, further comprising:
obtaining a vertical image range by the processor according to a vertical viewing angle and the depth of field length of the camera.

17. The augmented reality method of claim 16, further comprising:
obtaining a horizontal elongation by the processor according to the horizontal image range and an effective length of the needle.

18. The augmented reality method of claim 17, wherein
the effective length is greater than or equal to a sum of a first length of the first marking area and a second length of the second marking area.

19. The augmented reality method of claim 17, further comprising:
obtaining a vertical elongation by the processor according to the vertical image range and the effective length of the needle.

20. The augmented reality method of claim 19, further comprising:
obtaining an area elongation by the processor according to the horizontal image range, the vertical image range, and the effective length of the needle.
